(19) United States
(12) Patent Application Publication
(10) Pub. No.: US 2009/0054680 A1
(43) Pub. Date: Feb. 26, 2009

(54) SYNTHESIS OF 3,5,5-TRIMETHYL-2-CYCLOHEXENE SALICYLATE

(76) Inventors: Lowell J. Lawrence, Lexington, KY (US); Steven G. Mobley, Lexington, KY (US)

Correspondence Address: KING & SCHICKLI, PLLC, 247 NORTH BROADWAY, LEXINGTON, KY, 40507, US

(21) Appl. No.: 12/194,729

(22) Filed: Aug. 20, 2008

Related U.S. Application Data
(60) Provisional application No. 60/957,288, filed on Aug. 22, 2007.

(51) Int. Cl. C07C 67/08

(52) U.S. Cl. 560/75

(57) ABSTRACT

A process for preparing 3,5,5-Trimethyl-2-cyclohexene salicylate includes the reacting of 2-hydroxybenzoic acid with 3,5,5-Trimethyl-2-cyclohexen-1-ol in the presence of a coupling reagent.

This application claims the benefit of U.S. Provisional Patent Application Serial No. 60/957,288 filed 22 Aug. 2007.

### TECHNICAL FIELD

The present invention relates generally to ester synthesis methods and more specifically to a method for synthesizing 3,5,5-Trimethyl-2-cyclohexene salicylate

### BACKGROUND OF THE INVENTION

U.S. Pat. Nos. 6,060,062 to Fowler and 5,972,344, 5,989,559 and 6,013.260 all to Edwards clearly show that it is known in the art to utilize banana peel extract to relieve arthritic pain. The present invention relates to a method for preparing 3,5,5-Trimethyl-2-cyclohexene salicylate, an active ingredient in banana extract useful in relieving arthritic pain.

### SUMMARY OF THE INVENTION

In accordance with the purposes of the present invention as described herein a process is provided for preparing 3,5,5-Trimethyl-2-cyclohexene salicylate or 2-hydroxy benzoic acid 3,5,5-tiemthyl-cyclohex-2-enyl ester having a formula:

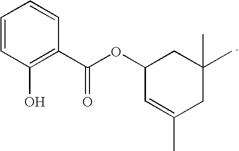

The process may be broadly described as comprising the step of reacting 2-hydroxybenzoic acid with 3,5,5-Trimethyl-2-cyclohexen-1-ol.

More specifically, the process includes performing the reacting step in the presence of a coupling reagent. That coupling reagent may be selected from a group of coupling reagents consisting of acyl halides, carbonic mixed anhydrides, carboxylic mixed anhydrides, phosphorous mixed anhydrides, sulfonic mixed anhydrides, carbodiimides, active ester reagents and mixtures thereof. Still more specifically, the coupling reagents may be selected from a group consisting of N-(3-Dimethylaminopropyl)-N′-ethylcarbodiimide hydrochloride, 1,3-dicyclocarbidimide, cyanuric chloride and mixtures thereof.

Alternatively, the process for preparing 3,5,5-Trimethyl-2-cyclohexene salicylate may be described as comprising the steps of dissolving 2-hydroxybenzoic acid in a solvent to form a reaction mixture, adding 3,5,5-Trimethyl-2-cyclohexen-1-ol to the reaction mixture, adding a coupling reagent to the reaction mixture, reacting the 2-hydroxybenzoic acid with the 3,5,5-Trimethyl-2-cyclohexen-1-ol to produce 3,5,5-Trimethyl-2-cyclohexene salicylate, quenching the reaction and recovering the 3,5,5-Trimethyl-2-cyclohexene salicylate.

The process further includes selecting the solvent from a group of solvents consisting of tetrahydrofuran, dioxane, acetonitrile, dichloromethane, dimethylformamide and mixtures thereof. The process further includes selecting a coupling reagent from a group of coupling reagents consisting of acyl halides, carbonic mixed anhydrides. carboxylic mixed anhydrides, phosphorous mixed anhydrides, sulfonic mixed anhydrides, carbodiimides, active ester reagents and mixtures thereof.

In addition, the process further includes the step of cooling the reaction mixture to about 0° C. before adding the 3,5,5-Trimethyl-2-cyclohexen-1-ol and the coupling reagent. In addition, the method includes stirring the reaction mixture during the reacting of the 2-hydroxybenzoic acid with the 3,5,5-Trimethyl-2-cyclohexen-1-ol.

Alternatively, the process may be described as comprising the steps of reacting 2-hyrdroxybenzoic acid with 3,5,5-Trimethyl-2-cyclohexen-1-ol in the presence of a coupling reagent to produce 3,5,5-Trimethyl-2-cyclohexene salicylate and purifying the 3,5,5-Trimethyl-2-cyclohexene salicylate by column chromatography or by distillation. The process includes the step of selecting the coupling reagent from a group consisting of acyl halides, carbonic mixed anhydrides, carboxylic mixed anhydrides, phosphorous mixed anhydrides, sulfonic mixed anhydrides, carbodiimides, active ester reagents and mixtures thereof. In one particularly useful embodiment the coupling reagent is cyanuric chloride. The method allows producing the 3,5,5-Trimethyl-2-cyclohexene salicylate as a racemic mixture of enantiomers, in either enantiomerically pure form or in any ratio in between. The enantiomerically pure compound may be prepared using an enantiomerically pure form of the 3,5,5-Trimethyl-2-cyclohexen-1-ol or by using a chiral column to separate the enantiomers of a racemic mixture.

### DETAILED DESCRIPTION OF THE PREFERRED EMBODIMENTS OF THE INVENTION

A process for preparing 3,5,5-Trimethyl-2-cyclohexene salicylate comprises reacting 2-hydroxybenzoic acid with 3,5,5-Trimethyl-2-cyclohexen-1-ol as illustrated below.

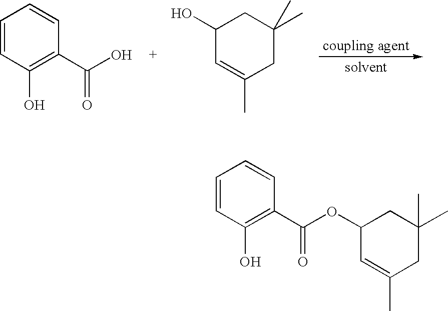

More specifically, the process comprises the steps of dissolving the 2-hydroxybenzoic acid in an appropriate solvent in order to form a reaction mixture. Solvents useful in the present invention include but are not limited to tetrahydrofuran, dioxane, acetonitrile, dichloromethane, dimethyl formamide and mixtures thereof. 3,5,5-Trimethyl-2-cyclohexen-1-ol and an appropriate coupling reagent are then added to the reaction mixture. Typically the reaction mixture is cooled to 0° C. in an ice bath prior to the addition of the 3,5,5-Trimethyl-2-cyclohexen-1-ol and the coupling reagent. Coupling reagents useful in the present invention include various classes of compounds such as acyl halides, carbonic mixed anhydrides, carboxylic mixed anhydrides, phosphorous mixed anhydrides, sulfonic mixed anhydrides, carbodiimides, active ester reagents and mixtures thereof. Typically the selected coupling agent is N-(3-Dimethylaminopropyl)-N′-ethylcarbodiimide hydrochloride, 1,3-dicyclocarbidimide, cyanuric chloride and mixtures thereof.

The reaction mixture is stirred during the reaction of the 2-hydroxybenzoic acid with the 3,5,5-Trimethyl-2-cyclohexen-1-ol. After the complete disappearance of the alcohol, the reaction is quenched and then extracted with an appropriate solvent such as dichloromethane, ethyl acetate or toluene and the resulting 3,5,5-Trimethyl-2-cyclohexene salicylate is then purified by column chromatography or by distillation. The distillation process comprises of removing the extraction solvent followed by vacuum distillation at 60° C. at 10-40 mbar. The 3,5,5-Trimethyl-2-cyclohexene salicylate may be prepared as a racemic mixture, or in its enantiomerically pure D or L form or in any ratio in between. Racemic mixtures may be prepared utilizing a racemic mixture of 3,5,5-Trimethyl-2-cyclohexen-1-ol as a starting material. The enantiomerically pure L form of 3,5,5-Trimethyl-2-cyclohexene salicylate may be prepared utilizing the L form of 3,5,5-Trimethyl-2-cyclohexen-1-ol as a starting material. Similarly, the enantiomerically pure R form of 3,5,5-Trimethyl-2-cyclohexene salicylate may be prepared by utilizing the enantiomerically pure R form of 3,5,5-Trimethyl-2-cyclohexen-1-ol as a starting material. Alternatively, a racemic mixture of 3,5,5-Trimethyl-2-cyclohexene salicylate may be resolved into the enantiomerically pure forms by using a chiral column for separation.

The following synthesis and example is presented to further illustrate the invention, but it is not to be considered as limited thereto. In the example, the 2-hydroxybenzoic acid and 3,5,5-Trimethyl-2-cyclohexen-1-ol starting materials were obtained from Sigma Aldrich Inc.

### EXAMPLE

2-hydroxybenzoic acid ( 1.38 g, 10 mmol) was dissolved in 20 mL of THF and cooled to 0° C. on ice bath. 3,5,5-Trimethyl-2-cyclohexen-1-ol (1.6 mL, 10.5 mmol) was added to this solution, followed by addition of N-(3-Dimethylaminopropyl)-N′-ethylcarbodiimide hydrochloride (2.0 g, 10.5 mmol). The reaction mixture was stirred on ice bath for 30 minutes then allowed to come to room temperature and stirred for 2 hours. After complete disappearance of reactants the reaction was quenched with 20 mL of 1 N sodium bicarbonate and extracted with dichloromethane (3×30 mL). The organic layers were combined, dried and evaporated to obtain an oily residue which was purified by column chromatography using silica as support and Hexane:Ethyl acetate (20:1) as eluant. The fractions were collected and evaporation of solvents gave product as clear oily product, which was analyzed using proton NMR, Massspec and HPLC.

The foregoing descriptions of the preferred embodiments of the present invention have been presented for purposes of illustration and description. It is not intended to be exhaustive or to limit the invention to the precise form disclosed. Obvious modifications or variations are possible in light of the above teachings. The embodiments were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled. The preferred embodiments do not and are not intended to limit the ordinary meaning of the claims in their fair and broad interpretation in any way.

What is claimed:

1. A process for preparing 3,5,5-Trimethyl-2-cyclohexene salicylate comprising:
reacting 2-hydroxybenzoic acid with 3,5,5-trimethyl-2-cyclohexen-1-ol.

2. The process of claim 1, including performing said reacting step in the presence of a coupling reagent selected from a group consisting of N-(3-Dimethylaminopropyl)-N′-ethylcarbodiimide hydrochloride, 1,3-dicyclocarbidimide, cyanuric chloride and mixtures thereof.

3. The process of claim 1, including performing said reacting step in the presence of a coupling reagent selected from a group consisting of acyl halides, carbonic mixed anhydrides, carbolic mixed anhydrides, phosphorous mixed anhydrides, sulfonic mixed anhydrides, carbodiimides, active ester reagents and mixtures thereof.

4. A process for preparing 3,5,5-Trimethyl-2-cyclohexene salicylate comprising:
dissolving 2-hydroxybenzoic acid in a solvent to form a reaction mixture;
adding a coupling reagent to said reaction mixture;
reacting said 2-hydroxybenzoic acid with said 3,5,5-trimethyl-2-cyclohexen-1-ol to produce 3,5,5-Trimethyl-2-cyclohexene salicylate;
quenching said reaction; and
recovering said 3,5,5-Trimethyl-2-cyclohexene salicylate.

5. The process of claim 4, including selecting said solvent from a group of solvents consisting of tetrahydrofuran, dioxane, acetonitrile, dichloromethane, dimethylformamide and mixtures thereof.

6. The process of claim 4, including selecting said coupling reagent from a group of coupling reagents consisting of acyl halides, carbonic mixed anhydrides, carbolic mixed anhydrides, phosphorous mixed anhydrides, sulfonic mixed anhydrides, carbodiimides, active ester reagents and mixtures thereof.

7. The process of claim 4, including cooling said reaction mixture to about 0° C. before adding said 3,5,5-trimethyl-2-cyclohexen-1-ol and said coupling reagent.

8. The process of claim 7, including stirring said reaction mixture during reacting of said 2-hydroxybenzoic acid with said 3,5,5-trimethyl-2-cyclohexen-1-ol.

9. A process for preparing 3,5,5-Trimethyl-2-cyclohexene salicylate comprising:
reacting 2-hydroxybenzoic acid with 3,5,5-trimethyl-2-cyclohexen-1-ol in the presence of a coupling reagent to produce 3,5,5-Trimethyl-2-cyclohexene salicylate; and
purifying said 3,5,5-Trimethyl-2-cyclohexene salicylate by column chromatography or by distillation.

10. The process of claim 9, wherein said coupling agent is selected from a group consisting of acyl halides, carbonic mixed anhydrides, carbolic mixed anhydrides, phosphorous mixed anhydrides, sulfonic mixed anhydrides, carbodiimides, active ester reagents and mixtures thereof.

11. The process of claim 9, wherein said coupling agent is cyanuric chloride.

12. The process of claim 10 including producing said 3,5,5-Trimethyl-2-cyclohexene salicylate as a racemic mixture of enantiomers.

13. The process of claim 10, including producing said 3,5,5-Trimethyl-2-cyclohexene salicylate in an enantiomerically pure form by using an enantiomerically pure form of 3,5,5-trimethyl-2-cyclohexen-1-ol.

14. The process of claim 10, including producing said 3,5,5-Trimethyl-2-cyclohexene salicylate in an enantiomerically pure form by separating a mixture of enantiomers using a chiral column.
